(19) United States
(12) Patent Application Publication
(10) Pub. No.: US 2024/0341770 A1
(43) Pub. Date: Oct. 17, 2024

(54) OCCLUSIVE IMPLANT SYSTEM

(71) Applicant: BOSTON SCIENTIFIC SCIMED,INC., MAPLE GROVE, MN (US)

(72) Inventors: Sara Reda Eskander, Plymouth, MN (US); David John Onushko, Maple Grove, MN (US); Anthony Frank Tassoni, JR., Andover, MN (US); Nicholas Lee Tassoni, Andover, MN (US)

(73) Assignee: BOSTON SCIENTIFIC SCIMED, INC., MAPLE GROVE, MN (US)

(21) Appl. No.: 18/631,575

(22) Filed: Apr. 10, 2024

Related U.S. Application Data
(60) Provisional application No. 63/458,541, filed on Apr. 11, 2023.

(51) Int. Cl. A61B 17/12; A61B 17/00

(52) U.S. Cl. CPC A61B 17/12122; A61B 17/00234; A61B 17/12031; A61B 17/12172; A61B 2017/00305; A61B 2017/00831; A61B 2017/1205

(57) ABSTRACT

A delivery system for an occlusive implant may include a delivery sheath having a delivery lumen extending proximally from a distal end of the delivery sheath, the delivery sheath being formed from a polymeric material, and a core wire slidably disposed within the delivery lumen. The core wire may be configured to releasably engage the occlusive implant. The delivery sheath may include a circumferential ring monolithically formed therewith at the distal end of the delivery sheath and a plurality of longitudinal slots disposed proximal of the circumferential ring. An occlusive implant system may include an occlusive implant configured to shift between a delivery configuration and a deployed configuration, and the delivery system for the occlusive implant. The occlusive implant may be disposed within a distal portion of the delivery lumen in the delivery configuration.

### CROSS-REFERENCE TO RELATED APPLICATIONS

This application claims the benefit of priority of U.S. Provisional Application No. 63/458,541 filed Apr. 11, 2023, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

In one example, a delivery system for an occlusive implant may comprise a delivery sheath having a delivery lumen extending proximally from a distal end of the delivery sheath, the delivery sheath being formed from a polymeric material, and a core wire slidably disposed within the delivery lumen, the core wire being configured to releasably engage the occlusive implant. The delivery sheath may include a circumferential ring monolithically formed therewith at the distal end of the delivery sheath and a plurality of longitudinal slots disposed proximal of the circumferential ring.

In addition or alternatively to any example disclosed herein, the plurality of longitudinal slots is disposed adjacent the circumferential ring.

In addition or alternatively to any example disclosed herein, the plurality of longitudinal slots extends partially through a side wall of the delivery sheath.

In addition or alternatively to any example disclosed herein, the plurality of longitudinal slots extends completely through a side wall of the delivery sheath.

In addition or alternatively to any example disclosed herein, a second polymeric material different from the polymeric material is disposed within at least one longitudinal slot of the plurality of longitudinal slots.

In addition or alternatively to any example disclosed herein, the plurality of longitudinal slots comprises a first plurality of longitudinal slots having a first length and a second plurality of longitudinal slots having a second length different from the first length.

In addition or alternatively to any example disclosed herein, the first plurality of longitudinal slots alternates with the second plurality of longitudinal slots around a circumference of the delivery sheath.

In addition or alternatively to any example disclosed herein, the plurality of longitudinal slots comprises a first circumferential row of longitudinal slots and a second circumferential row of longitudinal slots disposed proximal of the first circumferential row of longitudinal slots.

In addition or alternatively to any example disclosed herein, adjacent longitudinal slots of the second circumferential row of longitudinal slots are spaced apart circumferentially a greater distance than adjacent longitudinal slots of the first circumferential row of longitudinal slots.

In addition or alternatively to any example disclosed herein, the first circumferential row of longitudinal slots comprises a first quantity of longitudinal slots and the second circumferential row of longitudinal slots comprises a second quantity of longitudinal slots less than the first quantity of longitudinal slots.

In addition or alternatively to any example disclosed herein, each longitudinal slot of the second circumferential row of longitudinal slots is axially aligned with one longitudinal slot of the first circumferential row of longitudinal slots.

In addition or alternatively to any example disclosed herein, the delivery sheath further comprises a second circumferential ring monolithically formed with the delivery sheath. The second circumferential ring may be disposed axially between the first circumferential row of longitudinal slots and the second circumferential row of longitudinal slots.

In addition or alternatively to any example disclosed herein, at least one longitudinal slot of the plurality of longitudinal slots is wider at a distal end of the at least one longitudinal slot than at a proximal end of the at least one longitudinal slot.

In addition or alternatively to any example disclosed herein, the at least one longitudinal slot is tapered from the proximal end to the distal end.

In addition or alternatively to any example disclosed herein, an occlusive implant system may comprise an occlusive implant configured to shift between a delivery configuration and a deployed configuration, and a delivery system for the occlusive implant. The delivery system may comprise a delivery sheath having a delivery lumen extending proximally from a distal end of the delivery sheath, the delivery sheath being formed from a polymeric material, and a core wire slidably disposed within the delivery lumen, the core wire being releasably engaged with the occlusive implant at a distal end of the core wire. The delivery sheath may include a circumferential ring monolithically formed therewith at the distal end of the delivery sheath and a plurality of longitudinal slots disposed proximal of the circumferential ring. The occlusive implant may be disposed within a distal portion of the delivery lumen in the delivery configuration.

In addition or alternatively to any example disclosed herein, the occlusive implant comprises an expandable framework and an occlusive element secured to the expandable framework.

In addition or alternatively to any example disclosed herein, the distal end of the delivery sheath is configured to expand radially outward during recapture of the occlusive implant.

In addition or alternatively to any example disclosed herein, the circumferential ring extends uninterruptedly around the delivery lumen.

In addition or alternatively to any example disclosed herein, a delivery system for an occlusive implant may comprise a delivery sheath having a delivery lumen extending proximally from a distal end of the delivery sheath, the delivery sheath being formed from a polymeric material, and a core wire slidably disposed within the delivery lumen, the core wire being configured to releasably engage the occlusive implant. The delivery sheath may include a circumferential ring monolithically formed therewith at the distal end of the delivery sheath, and a plurality of longitudinal strips formed from a second polymeric material different from the polymeric material. The plurality of longitudinal strips may be embedded within a side wall of the delivery sheath proximal of the circumferential ring.

In addition or alternatively to any example disclosed herein, the second polymeric material has a lower durometer than the polymeric material.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 are side views of selected aspects of an occlusive implant system;

FIG. 3 illustrates selected aspects of a configuration of a delivery system for an occlusive implant of the occlusive implant system of FIGS. 1-2;

FIG. 4A is a cross-sectional view illustrating selected aspects of a configuration of the delivery system of FIG. 3;

FIG. 4B is a cross-sectional view illustrating selected aspects of a configuration of the delivery system of FIG. 3;

FIG. 5 illustrates selected aspects of a delivery system for an occlusive implant of the occlusive implant system of FIGS. 1-2;

FIG. 6 is a cross-sectional view illustrating selected aspects of a configuration of the delivery system of FIG. 5;

FIG. 7 illustrates selected aspects of a delivery system for an occlusive implant of the occlusive implant system of FIGS. 1-2;

FIG. 8 illustrates selected aspects of a delivery system for an occlusive implant of the occlusive implant system of FIGS. 1-2;

FIG. 9 illustrates selected aspects of a delivery system for an occlusive implant of the occlusive implant system of FIGS. 1-2;

FIG. 10 illustrates selected aspects of a delivery system for an occlusive implant of the occlusive implant system of FIGS. 1-2;

FIG. 11 is a cross-sectional view illustrating selected aspects of a configuration of the delivery system of FIG. 10; and

FIGS. 12-13 schematically illustrate selected aspects of recapturing an occlusive implant using a delivery system according to the disclosure.

While aspects of the disclosure are amenable to various modifications and alternative forms, examples are shown in the drawings and described herein. It should be understood, however, that the intention is not to limit aspects of the disclosure to the particular embodiments described. On the contrary, the disclosure shall cover all modifications, equivalents, and alternatives falling within the spirit and scope thereof.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

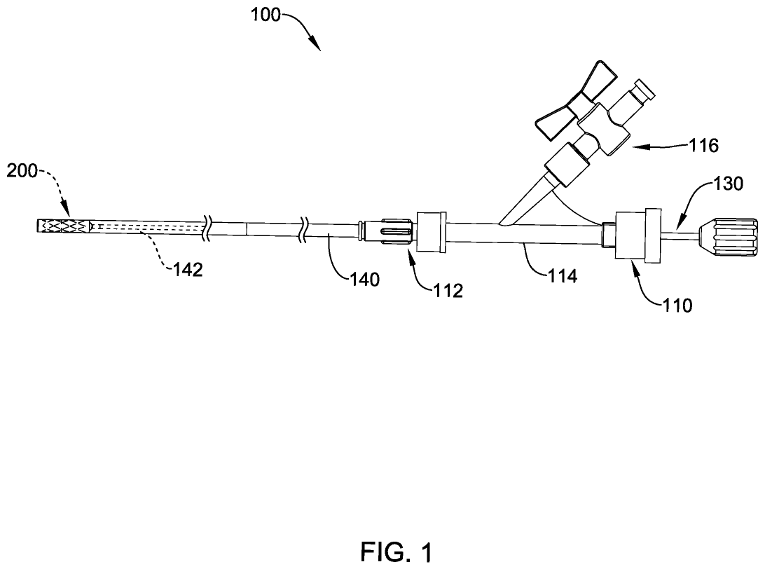

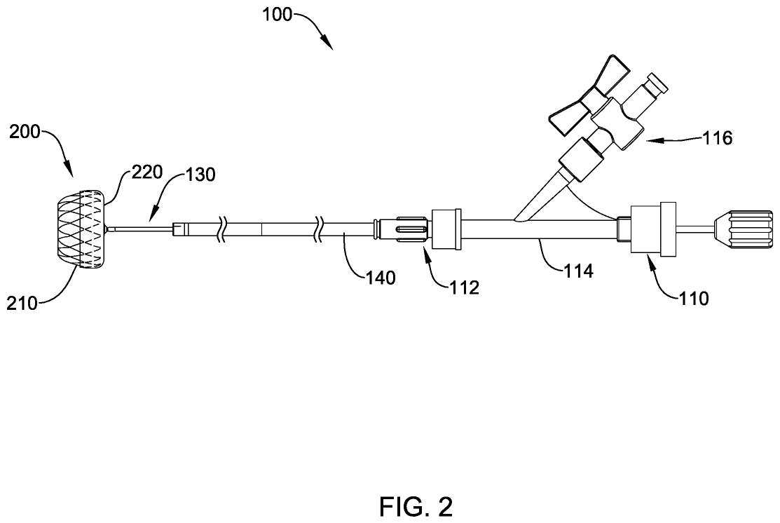

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 100 including a delivery sheath 140 having a delivery lumen 142 extending proximally from a distal end of the delivery sheath 140. In one example, the delivery lumen 142 extends from a proximal opening to a distal opening of the delivery sheath 140. The delivery system 100 may include a proximal hub 110. In some embodiments, the delivery system may include a mid-hub 112. In some embodiments, the delivery system 100 may include a mid-shaft 114 extending from the proximal hub 110 to the mid-hub 112. In some embodiments, the delivery sheath 140 may extend distally from the mid-hub 112. Other configurations are also contemplated. In some embodiments, the delivery system 100 may include a side port 116. In some embodiments, the side port 116 may be in communication with the mid-shaft 114. Other configurations are also contemplated. In some embodiments, the delivery system 100 and/or the delivery lumen 142 may include a proximal segment (not shown) extending within and/or through the mid-hub 112, the mid-shaft 114, and the proximal hub 110. In some embodiments, the proximal segment may be in fluid communication with and/or may be an extension of the delivery lumen 142 of the delivery sheath 140. In some embodiments, the side port 116 may be in fluid communication with the proximal segment and/or the delivery lumen 142.

The occlusive implant system and/or the delivery system 100 may include a core wire 130 slidably and/or rotatably disposed within the delivery lumen 142 (and the proximal segment, where present). The occlusive implant system may include an occlusive implant 200, which may be configured for implantation within a left atrial appendage, releasably engaged with and/or releasably attached to a distal end of the core wire 130. In at least some embodiments, the occlusive implant 200 may be a left atrial appendage closure device. In some embodiments, a proximal end of the core wire 130 may extend proximally of a proximal end of the delivery sheath 140 and/or the proximal opening of the delivery lumen 142 for manual manipulation by a clinician or practitioner. In at least some embodiments, the delivery sheath 140 may comprise and/or may be formed from a polymeric material. In some embodiments, the delivery sheath 140 may comprise and/or may be formed from a plurality of polymeric materials. In some embodiments, the delivery sheath may comprise and/or may be formed from a combination of metallic and polymeric materials. In some embodiments, the delivery sheath 140 may include a reinforcing element, such as a mesh, a coil, a braid, etc., formed therein, embedded therein, attached thereto, etc. along at least a portion of a length of the delivery sheath 140. Other configurations are also contemplated. Some suitable, but non-limiting, examples of materials for the occlusive implant system, the core wire 130, and/or the delivery sheath 140, etc., including but not limited to metallic materials, polymeric materials, etc., are discussed below.

The occlusive implant 200 may include an expandable framework 210 (e.g., FIG. 2) configured to shift between a delivery configuration (e.g., FIG. 1), such as when the occlusive implant 200 is disposed within the delivery lumen 142 proximate the distal opening and/or within a distal portion of the delivery lumen 142, and a deployed configuration (e.g., FIG. 2) when the occlusive implant 200 is unconstrained by the delivery sheath 140.

In some embodiments, the expandable framework 210 may comprise a plurality of interconnected struts. In some embodiments, the expandable framework 210 may be compliant or semi-compliant and may generally conform to and/or be configured to sealingly engage with the shape and/or geometry of the left atrial appendage in the deployed configuration.

In some embodiments, a proximal end of the expandable framework 210 may be configured to relcasably attach, join, couple, engage, or otherwise connect to the distal end of the core wire 130 (e.g., FIG. 2). In some embodiments, the proximal end of the expandable framework 210 may include a proximal hub coupled and/or non-releasably attached thereto. In some embodiments, the proximal hub may be configured to and/or adapted to releasably couple with, join to, mate with, or otherwise engage a distal end of the core wire 130. Other means of releasably coupling and/or engaging the expandable framework 210 to the distal end of the core wire 130 are also contemplated.

In some embodiments, the occlusive implant 200 may include an occlusive element 220 (e.g., a membrane, a fabric, or a tissue element, etc.) connected to, disposed on, disposed over, disposed about, or covering at least a portion the expandable framework 210. In some embodiments, the occlusive element 220 may be connected to, disposed on, disposed over, disposed about, or cover at least a portion of an outer (or outwardly facing) surface of the expandable framework 210.

In some embodiments, the occlusive element 220 may be permeable or impermeable to blood and/or other fluids, such as water. In some embodiments, the occlusive element 220 may include a polymeric membrane, a metallic or polymeric mesh, a porous or semi-porous filter-like material, or other suitable construction. In some embodiments, the occlusive element 220 prevents thrombi (e.g., blood clots, etc.) from passing through the occlusive element 220 and out of the left atrial appendage into the blood stream. In some embodiments, the occlusive element 220 promotes endothelization after implantation, thereby effectively removing the target site (e.g., the left atrial appendage, etc.) from the patient's circulatory system. Some suitable, but non-limiting, examples of materials for the occlusive element 220 are discussed below.

In some embodiments, the expandable framework 210 and/or the plurality of interconnected struts may be integrally formed and/or cut from a unitary member. In some embodiments, the expandable framework 210 and/or the plurality of interconnected struts may be integrally formed and/or cut from a unitary tubular member and subsequently formed and/or heat set to a desired shape in the deployed configuration. In some embodiments, the expandable framework 210 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 210 include laser cutting, machining, punching, stamping, electro discharge machining (EDM), chemical dissolution, etc. Other means and/or methods are also contemplated.

In use, the delivery sheath 140 may be advanced and/or navigated to the left atrial appendage to deliver the occlusive implant 200 thereto. In one example, the delivery sheath 140 may be advanced and/or navigated to the left atrial appendage using and/or over a guidewire. For example, the delivery sheath 140 may be advanced to the patient's left atrium and the distal end disposed adjacent to the left atrial appendage with the occlusive implant 200 disposed therein in the delivery configuration. In some embodiments, the delivery sheath 140 may include steering capability. After the distal end of the delivery sheath 140 is disposed adjacent to and/or at the left atrial appendage, the core wire 130 may be advanced distally relative to the delivery sheath 140 to advance the occlusive implant 200 out of the delivery sheath 140, where the occlusive implant 200 may shift to the deployed configuration.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 100 may be inserted through the access device. In some embodiments, the length of the delivery sheath 140 may be substantially equal to the length of the access device. In some embodiments, the length of the delivery sheath 140 may be slightly longer than the access device. During use, the delivery sheath 140 may be advanced within the access device with the occlusive implant 200 disposed therein in the delivery configuration. After the distal end of the delivery sheath 140 is disposed adjacent to and/or at the distal end of the access device, the core wire 130 may be advanced distally relative to the delivery sheath 140 and/or the access device to advance the occlusive implant 200 out of the delivery sheath 140 and the access device, where the occlusive implant 200 may shift to the deployed configuration.

In some embodiments, the delivery system, the delivery sheath 140, and/or the access device may be sized in accordance with its intended use. For example, the delivery system, the delivery sheath 140, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system, the delivery sheath 140, and/or the access device may vary based on the use or application. In some examples, the outer diameter of the delivery system, the delivery sheath 140, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system, the delivery sheath 140, and/or the access device may be a maximum of 5.66 mm (17 French) and is preferably smaller than 5.66 mm (17 French). Other configurations are also contemplated. In some embodiments, it is desirable for the outer diameter of the delivery system, the delivery sheath 140, and/or the access device to be as small as possible.

In some embodiments, during a procedure, it may be necessary to recapture the occlusive implant 200. For example, the initial placement of the occlusive implant 200 may be incorrect and/or inadequate. Accordingly, the delivery sheath 140 and/or the access device may be configured to permit recapture of the occlusive implant 200.

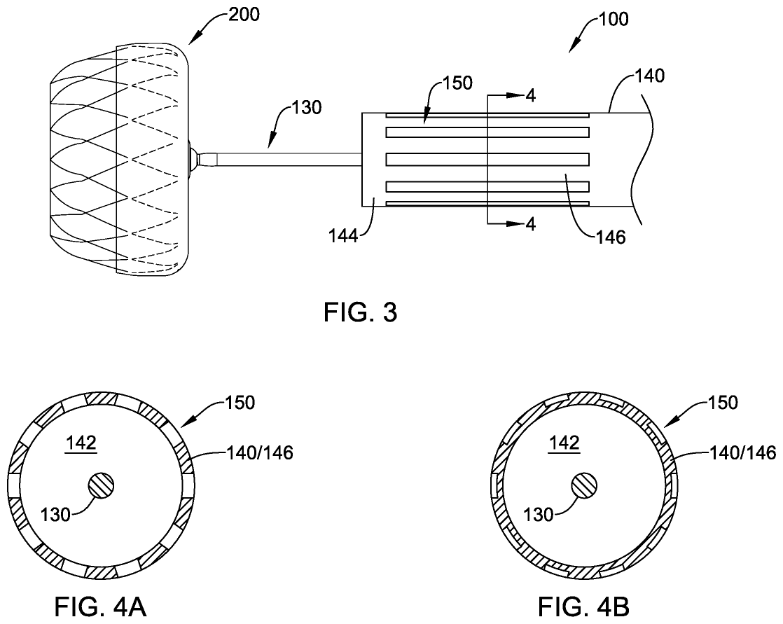

FIGS. 3-11 illustrate selected aspects of configurations of the delivery sheath 140 which may permit the delivery sheath 140 to recapture the occlusive implant 200 and/or may enhance a capability of the delivery sheath 140 to recapture the occlusive implant 200. For reference and illustrative purposes only, the core wire 130 and the occlusive implant 200 are shown in some figures. Additionally, some features are not shown in all drawings and some drawings may identify some features for reference that are not explicitly described or discussed with respect to a particular drawing. While described with reference to the delivery sheath 140, it shall be understood that the illustrated configurations may be applied to the access device in place of, or in addition to, the delivery sheath 140.

In some embodiments, the distal end and/or a distal end region of the delivery sheath 140 may be configured to resiliently expand radially outward during recapture of the occlusive implant 200. By permitting the distal end and/or the distal end region of the delivery sheath 140 to resiliently expand radially outward during recapture of the occlusive implant 200, damage to the occlusive implant 200 and/or the delivery sheath 140 may be avoided. Additionally, permitting the distal end and/or the distal end region of the delivery sheath 140 to resiliently expand radially outward during recapture of the occlusive implant 200 may reduce forces required to recapture the occlusive implant 200.

In some embodiments, the distal end region may be devoid of any reinforcing element(s). In some embodiments, the delivery sheath 140 may include a circumferential ring 144 monolithically formed therewith at the distal end of the delivery sheath 140. In some embodiments, the circumferential ring 144 may be disposed at a distalmost end of the delivery sheath 140 and/or may be a distalmost extent of the delivery sheath 140. In at least some embodiments, the circumferential ring 144 may extend continuously and/or uninterruptedly around the delivery lumen 142 and/or a circumference of the delivery sheath 140.

In some embodiments, the delivery sheath 140 may include a plurality of longitudinal slots 150 disposed and/or formed in a side wall 146 of the delivery sheath 140. The plurality of longitudinal slots 150 may be formed as openings and/or apertures within the side wall 146 of the delivery sheath 140. As such, the plurality of longitudinal slots 150 may be understood to be voids formed in portions of the side wall 146 or portions of the side wall 146 that are devoid of the material forming the side wall 146. In some embodiments, the plurality of longitudinal slots 150 may be formed by removing material from the side wall 146 of the delivery sheath 140. Other configurations and/or ways of making the plurality of longitudinal slots 150 are also contemplated.

In some embodiments, the plurality of longitudinal slots 150 may comprise two longitudinal slots, three longitudinal slots, four longitudinal slots, five longitudinal slots, six longitudinal slots, seven longitudinal slots, eight longitudinal slots, nine longitudinal slots, ten longitudinal slots, eleven longitudinal slots, twelve longitudinal slots, thirteen longitudinal slots, fourteen longitudinal slots, fifteen longitudinal slots, sixteen longitudinal slots, seventeen longitudinal slots, eighteen longitudinal slots, nineteen longitudinal slots, twenty longitudinal slots, or another suitable or desired number of longitudinal slots.

In some embodiments, the plurality of longitudinal slots 150 may have an axial length between about 0.127 millimeters (0.005 inches) and about 50.8 millimeters (2.0 inches). In some embodiments, the plurality of longitudinal slots 150 may have an axial length between about 0.1905 millimeters (0.0075 inches) and about 38.1 millimeters (1.5 inches). In some embodiments, the plurality of longitudinal slots 150 may have an axial length between about 0.254 millimeters (0.01 inches) and about 25.4 millimeters (1.0 inches). In some embodiments, the plurality of longitudinal slots 150 may have an axial length between about 0.508 millimeters (0.02 inches) and about 19.05 millimeters (0.75 inches). Other axial lengths are also contemplated.

In some embodiments, the plurality of longitudinal slots 150 may have a lateral width and/or a circumferential width between about 0.0127 millimeters (0.0005 inches) and about 12.7 millimeters (0.5 inches). In some embodiments, the plurality of longitudinal slots 150 may have a lateral width and/or a circumferential width between about 0.01905 millimeters (0.00075 inches) and about 6.35 millimeters (0.25 inches). In some embodiments, the plurality of longitudinal slots 150 may have a lateral width and/or a circumferential width between about 0.0254 millimeters (0.001 inches) and about 5.08 millimeters (0.2 inches). In some embodiments, the plurality of longitudinal slots 150 may have a lateral width and/or a circumferential width between about 0.0508 millimeters (0.002 inches) and about 2.54 millimeters (0.1 inches). Other lateral widths and/or circumferential widths are also contemplated.

In some embodiments, the circumferential ring 144 may have an axial length between about 0.127 millimeters (0.005 inches) and about 50.8 millimeters (2.0 inches). In some embodiments, the circumferential ring 144 may have an axial length between about 0.1905 millimeters (0.0075 inches) and about 38.1 millimeters (1.5 inches). In some embodiments, the circumferential ring 144 may have an axial length between about 0.254 millimeters (0.01 inches) and about 25.4 millimeters (1.0 inches). In some embodiments, the circumferential ring 144 may have an axial length between about 0.508 millimeters (0.02 inches) and about 19.05 millimeters (0.75 inches). Other axial lengths are also contemplated.

In some embodiments, the plurality of longitudinal slots 150 may be disposed proximal of the circumferential ring 144. In some embodiments, the plurality of longitudinal slots 150 may be spaced apart from the distal end of the delivery sheath 140. In some embodiments, the plurality of longitudinal slots 150 may be disposed adjacent to the circumferential ring 144. In some embodiments, the plurality of longitudinal slots 150 may be disposed immediately adjacent to the circumferential ring 144.

In some embodiments, the plurality of longitudinal slots 150 may be disposed around and/or about a central longitudinal axis of the delivery sheath 140. In some embodiments, the plurality of longitudinal slots 150 may be arranged and/or disposed in an array extending circumferentially around and/or about the delivery sheath 140 and/or the central longitudinal axis of the delivery sheath 140. The plurality of longitudinal slots 150 may be circumferentially spaced apart from each other.

In some embodiments, the plurality of longitudinal slots 150 may extend completely through the side wall 146 of the delivery sheath 140, as seen in FIG. 4A. In some embodiments, the plurality of longitudinal slots 150 may extend from an outer surface of the delivery sheath 140 to an inner surface of the delivery sheath 140 and/or the delivery lumen 142. In some embodiments, the delivery lumen 142 may be in fluid communication with an exterior of the delivery sheath 140 through the plurality of longitudinal slots 150.

In some embodiments, the plurality of longitudinal slots 150 may extend partially through the side wall 146 of the delivery sheath 140, as seen in FIG. 4B. In some embodiments, the plurality of longitudinal slots 150 may extend radially inward from the outer surface of the delivery sheath 140. In some alternative configurations, the plurality of longitudinal slots 150 may extend radially outward from the inner surface of the delivery sheath 140. In some embodiments, the plurality of longitudinal slots 150 may include longitudinal slots that extend radially inward from the outer surface of the delivery sheath 140 and longitudinal slots that extend radially outward from the inner surface of the delivery sheath 140. Other configurations and/or combinations thereof are also contemplated.

In some embodiments, the plurality of longitudinal slots 150 may extend through about 10% of the side wall 146 of the delivery sheath 140. In some embodiments, the plurality of longitudinal slots 150 may extend through about 20% of the side wall 146 of the delivery sheath 140. In some embodiments, the plurality of longitudinal slots 150 may extend through about 30% of the side wall 146 of the delivery sheath 140. In some embodiments, the plurality of longitudinal slots 150 may extend through about 40% of the side wall 146 of the delivery sheath 140. In some embodiments, the plurality of longitudinal slots 150 may extend through about 50% of the side wall 146 of the delivery sheath 140. In some embodiments, the plurality of longitudinal slots 150 may extend through about 60% of the side wall 146 of the delivery sheath 140. In some embodiments, the plurality of longitudinal slots 150 may extend through about 70% of the side wall 146 of the delivery sheath 140. In some embodiments, the plurality of longitudinal slots 150 may extend through about 80% of the side wall 146 of the delivery sheath 140. In some embodiments, the plurality of longitudinal slots 150 may extend through about 90% of the side wall 146 of the delivery sheath 140. Other configurations are also contemplated.

In some embodiments, various combinations of longitudinal slots that extend completely through the side wall 146 and longitudinal slots that extend partially through the side wall 146 are also contemplated. In some embodiments, the combinations of longitudinal slots that extend completely through the side wall 146 and longitudinal slots that extend partially through the side wall 146 may include longitudinal slots that extend varying amounts through the side wall 146 (e.g., some longitudinal slots extend completely through the side wall 146, some longitudinal slots extend a first percentage through the side wall 146, some longitudinal slots extend a second percentage through the side wall 146, etc.).

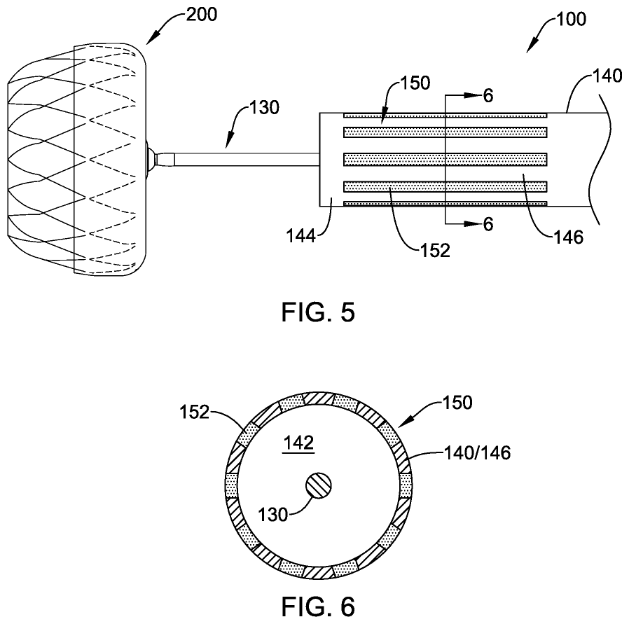

As discussed herein, in some embodiments, the side wall 146 and/or the delivery sheath 140 may be formed from a polymeric material. In some embodiments, a second polymeric material 152 different from the polymeric material may be disposed within at least one longitudinal slot of the plurality of longitudinal slots 150, as seen in FIGS. 5-6. In some embodiments, the second polymeric material 152 may be a softer material and/or a lower durometer material than the polymeric material. In some embodiments, the second polymeric material 152 may be more elastic and/or may stretch or elongate farther than the polymeric material without breaking and/or failing. In some embodiments, the second polymeric material 152 may be disposed in each longitudinal slot of the plurality of longitudinal slots 150. In some embodiments, the second polymeric material 152 may be disposed in a subset of the plurality of longitudinal slots 150 or in only some of the plurality of longitudinal slots 150. In some embodiments, the second polymeric material 152 may be disposed in only one longitudinal slot of the plurality of longitudinal slots 150.

In some embodiments, the second polymeric material 152 may extend completely through the side wall 146 of the delivery sheath 140, as seen in FIG. 6. In some embodiments, the second polymeric material 152 may extend from an outer surface of the delivery sheath 140 to an inner surface of the delivery sheath 140 and/or the delivery lumen 142. In some embodiments, the delivery lumen 142 may be prevented from being in fluid communication with an exterior of the delivery sheath 140 by the second polymeric material 152.

In some embodiments, the second polymeric material 152 may extend partially through the side wall 146 of the delivery sheath 140. In some embodiments, the second polymeric material 152 may extend radially inward from the outer surface of the delivery sheath 140. In some alternative configurations, the second polymeric material 152 may extend radially outward from the inner surface of the delivery sheath 140. In some embodiments, the second polymeric material 152 may be disposed between the inner surface of the delivery sheath 140 and the outer surface of the delivery sheath 140. In some embodiments, an inward facing surface of the second polymeric material 152 may be radially spaced apart from the inner surface of the delivery sheath 140 and/or an outward facing surface of the second polymeric material 152 may be radially spaced apart from the outer surface of the delivery sheath 140. Other configurations and/or combinations thereof are also contemplated.

In some embodiments, the second polymeric material 152 may have a radial thickness that is about 10% of a thickness of the side wall 146 of the delivery sheath 140. In some embodiments, the second polymeric material 152 may have a radial thickness that is about 20% of the thickness of the side wall 146 of the delivery sheath 140. In some embodiments, the second polymeric material 152 may have a radial thickness that is about 30% of the thickness of the side wall 146 of the delivery sheath 140. In some embodiments, the second polymeric material 152 may have a radial thickness that is about 40% of the thickness of the side wall 146 of the delivery sheath 140. In some embodiments, the second polymeric material 152 may have a radial thickness that is about 50% of the thickness of the side wall 146 of the delivery sheath 140. In some embodiments, the second polymeric material 152 may have a radial thickness that is about 60% of the thickness of the side wall 146 of the delivery sheath 140. In some embodiments, the second polymeric material 152 may have a radial thickness that is about 70% of the thickness of the side wall 146 of the delivery sheath 140. In some embodiments, the second polymeric material 152 may have a radial thickness that is about 80% of the thickness of the side wall 146 of the delivery sheath 140. In some embodiments, the second polymeric material 152 may have a radial thickness that is about 90% of the thickness of the side wall 146 of the delivery sheath 140. Other configurations are also contemplated.

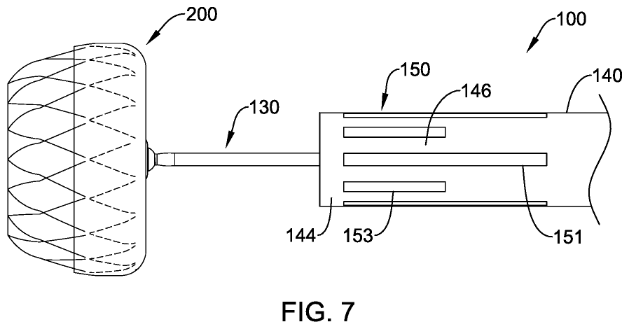

In some embodiments, the plurality of longitudinal slots 150 may comprise a first plurality of longitudinal slots 151 having a first length (e.g., a first longitudinal length or a first axial length) and a second plurality of longitudinal slots 153 having a second length (e.g., a second longitudinal length or a second axial length) different from the first length, as seen in FIG. 7. In at least some embodiments, the second length may be less than the first length. In some embodiments, the plurality of longitudinal slots 150 may comprise additional pluralities of longitudinal slots having lengths different from the first length and/or the second length (e.g., a third plurality of longitudinal slots having a third length, etc.).

In some embodiments, the first plurality of longitudinal slots 151 may alternate with the second plurality of longitudinal slots 153 around the circumference of the delivery sheath 140. Other patterns and/or distributions of the first plurality of longitudinal slots 151 and/or the second plurality of longitudinal slots 153 around the circumference of the delivery sheath 140 are also contemplated. In one non-limiting example, the first plurality of longitudinal slots 151 may be arranged in pairs with a single longitudinal slot of the second plurality of longitudinal slots 153 disposed between adjacent pairs of the first plurality of longitudinal slots 151 (e.g., a “two-one-two” configuration) around the circumference of the delivery sheath 140. Other examples may include but are not limited to a “three-one-three” configuration, a “two-two-two” configuration, a “one-three-one” configuration, etc.

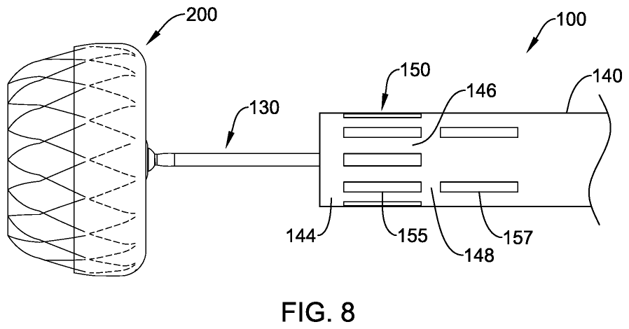

In some embodiments, the plurality of longitudinal slots 150 may comprise a first circumferential row of longitudinal slots 155 and a second circumferential row of longitudinal slots 157 disposed proximal of the first circumferential row of longitudinal slots 155, as seen in FIG. 8. The first circumferential row of longitudinal slots 155 may be axially and/or longitudinally spaced apart from the second circumferential row of longitudinal slots 157. In some embodiments, the delivery sheath 140 may comprise a second circumferential ring 148 monolithically formed with the delivery sheath 140. In some embodiments, the second circumferential ring 148 may be disposed proximal of the circumferential ring 144 and/or the first circumferential row of longitudinal slots 155. In some embodiments, the second circumferential ring 148 may be axially disposed between the first circumferential row of longitudinal slots 155 and the second circumferential row of longitudinal slots 157. In at least some embodiments, the second circumferential ring 148 may extend continuously and/or uninterruptedly around the delivery lumen 142 and/or a circumference of the delivery sheath 140.

In some embodiments, the second circumferential ring 148 may have a longitudinal dimension or a longitudinal extent that is similar to a longitudinal dimension or a longitudinal extent of the circumferential ring 144. In some embodiments, the longitudinal dimension or the longitudinal extent of the second circumferential ring 148 may be equal to the longitudinal dimension or the longitudinal extent of the circumferential ring 144. In some embodiments, the longitudinal dimension or the longitudinal extent of the second circumferential ring 148 may be less than the longitudinal dimension or the longitudinal extent of the circumferential ring 144. In some embodiments, the longitudinal dimension or the longitudinal extent of the second circumferential ring 148 may be greater than the longitudinal dimension or the longitudinal extent of the circumferential ring 144.

Circumferentially adjacent longitudinal slots of the first circumferential row of longitudinal slots 155 may be spaced apart circumferentially from each other around the circumference of the delivery sheath 140. Circumferentially adjacent longitudinal slots of the second circumferential row of longitudinal slots 157 may be spaced apart circumferentially from each other around the circumference of the delivery sheath 140. In some embodiments, circumferentially adjacent longitudinal slots of the second circumferential row of longitudinal slots 157 may be spaced apart circumferentially a greater distance than circumferentially adjacent longitudinal slots of the first circumferential row of longitudinal slots 155.

In some embodiments, the first circumferential row of longitudinal slots 155 may comprise a first quantity of longitudinal slots and the second circumferential row of longitudinal slots 157 may comprise a second quantity of longitudinal slots less than the first quantity of longitudinal slots. For example, the first circumferential row of longitudinal slots 155 may have more longitudinal slots extending around the circumference of the delivery sheath 140 than the second circumferential row of longitudinal slots 157. In general, it may be beneficial for the delivery sheath 140 to have more “open space” in, cut out area in, voids in, or material removed from the side wall 146 around its circumference at locations closer to the distal end of the delivery sheath 140. Accordingly, it is also contemplated that the first quantity of longitudinal slots may be less than the second quantity of longitudinal slots with the additional condition or characteristic that longitudinal slots of the first circumferential row of longitudinal slots 155 are larger or circumferentially wider than longitudinal slots of the second circumferential row of longitudinal slots 157 (e.g., more of the side wall 146 remains between the longitudinal slots of the second circumferential row of longitudinal slots 157 than between the longitudinal slots of the first circumferential row of longitudinal slots 155).

In some embodiments, each longitudinal slot of the second circumferential row of longitudinal slots 157 may be axially aligned with one longitudinal slot of the first circumferential row of longitudinal slots 155. In some embodiments, each longitudinal slot of the second circumferential row of longitudinal slots 157 may cooperate and/or combine with one longitudinal slot of the first circumferential row of longitudinal slots 155 to form longer longitudinal slots interrupted by the second circumferential ring 148. In some embodiments, the longer longitudinal slots interrupted by the second circumferential ring 148 may alternate around the circumference of the delivery sheath 140 with shorter longitudinal slots formed by remaining slots of the first circumferential row of longitudinal slots 155 not included in or used to form the longer longitudinal slots interrupted by the second circumferential ring 148.

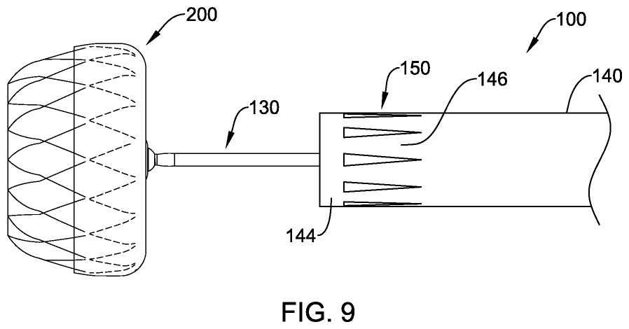

In some embodiments, at least one longitudinal slot of the plurality of longitudinal slots 150 may be wider at a distal end of the at least one longitudinal slot than at a proximal end of the at least one longitudinal slot. In some embodiments, the at least one longitudinal slot of the plurality of longitudinal slots 150 may be stepped from the proximal end toward and/or to the distal end. In some embodiments, the at least one longitudinal slot of the plurality of longitudinal slots 150 may be tapered from the proximal end toward and/or to the distal end, as seen in FIG. 9. Other configurations are also contemplated.

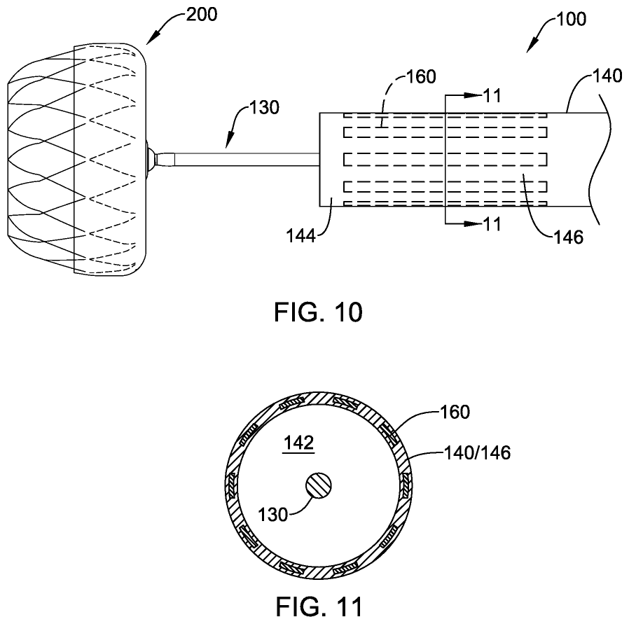

In some alternative embodiments, the delivery sheath 140 may include the circumferential ring 144 monolithically formed therewith at the distal end of the delivery sheath 140, and a plurality of longitudinal strips 160 formed from a second polymeric material different from the polymeric material forming the delivery sheath 140 and/or the side wall 146 of the delivery sheath 140. The plurality of longitudinal strips 160 may be embedded within the side wall 146 of the delivery sheath 140 proximal of the circumferential ring 144, as seen in FIGS. 10-11. The second polymeric material forming the plurality of longitudinal strips 160 may be a softer material and/or a lower durometer material than the polymeric material. In some embodiments, the second polymeric material may be more elastic and/or may stretch or elongate farther than the polymeric material without breaking and/or failing.

In some embodiments, the plurality of longitudinal strips 160 may comprise a first plurality of longitudinal strips having a first length (e.g., a first longitudinal length or a first axial length) and a second plurality of longitudinal strips having a second length (e.g., a second longitudinal length or a second axial length) different from the first length, similar to the plurality of longitudinal slots 150 of FIG. 7 above. In at least some embodiments, the second length may be less than the first length. In some embodiments, the plurality of longitudinal strips 160 may comprise additional pluralities of longitudinal slots having lengths different from the first length and/or the second length (e.g., a third plurality of longitudinal slots having a third length, etc.).

In some embodiments, the first plurality of longitudinal strips may alternate with the second plurality of longitudinal strips around the circumference of the delivery sheath 140. Other patterns and/or distributions of the first plurality of longitudinal strips and/or the second plurality of longitudinal strips around the circumference of the delivery sheath 140 are also contemplated. In one non-limiting example, the first plurality of longitudinal strips may be arranged in pairs with a single longitudinal slot of the second plurality of longitudinal strips disposed between adjacent pairs of the first plurality of longitudinal strips (e.g., a “two-one-two” configuration) around the circumference of the delivery sheath 140. Other examples may include but are not limited to a “three-one-three” configuration, a “two-two-two” configuration, a “one-three-one” configuration, etc.

In some embodiments, the plurality of longitudinal strips 160 may comprise a first circumferential row of longitudinal strips and a second circumferential row of longitudinal strips disposed proximal of the first circumferential row of longitudinal strips, similar to the plurality of longitudinal slots 150 of FIG. 8 above. The first circumferential row of longitudinal strips may be axially and/or longitudinally spaced apart from the second circumferential row of strips. In some embodiments, the delivery sheath 140 may comprise a second circumferential ring, similar to the second circumferential ring 148 above, monolithically formed with the delivery sheath 140 and disposed proximal of the circumferential ring 144 and/or the first circumferential row of longitudinal strips. In some embodiments, the second circumferential ring may be axially disposed between the first circumferential row of longitudinal strips and the second circumferential row of longitudinal strips. In at least some embodiments, the second circumferential ring may extend continuously and/or uninterruptedly around the delivery lumen 142 and/or a circumference of the delivery sheath 140.

In some embodiments, the second circumferential ring may have a longitudinal dimension or a longitudinal extent that is similar to a longitudinal dimension or a longitudinal extent of the circumferential ring 144. In some embodiments, the longitudinal dimension or the longitudinal extent of the second circumferential ring may be equal to the longitudinal dimension or the longitudinal extent of the circumferential ring 144. In some embodiments, the longitudinal dimension or the longitudinal extent of the second circumferential ring may be less than the longitudinal dimension or the longitudinal extent of the circumferential ring 144. In some embodiments, the longitudinal dimension or the longitudinal extent of the second circumferential ring may be greater than the longitudinal dimension or the longitudinal extent of the circumferential ring 144.

Circumferentially adjacent longitudinal slots of the first circumferential row of longitudinal strips may be spaced apart circumferentially from each other around the circumference of the delivery sheath 140. Circumferentially adjacent longitudinal slots of the second circumferential row of longitudinal strips may be spaced apart circumferentially from each other around the circumference of the delivery sheath 140. In some embodiments, circumferentially adjacent longitudinal slots of the second circumferential row of longitudinal strips may be spaced apart circumferentially a greater distance than circumferentially adjacent longitudinal slots of the first circumferential row of longitudinal strips.

In some embodiments, the first circumferential row of longitudinal strips may comprise a first quantity of longitudinal strips and the second circumferential row of longitudinal strips may comprise a second quantity of longitudinal strips less than the first quantity of longitudinal strips. For example, the first circumferential row of longitudinal strips may have more longitudinal strips extending around the circumference of the delivery sheath 140 than the second circumferential row of longitudinal strips. In general, it may be beneficial for the delivery sheath 140 to have more of the second polymeric material at locations closer to the distal end of the delivery sheath 140. Accordingly, it is also contemplated that the first quantity of longitudinal strips may be less than the second quantity of longitudinal strips with the additional condition or characteristic that longitudinal slots of the first circumferential row of longitudinal strips are larger or circumferentially wider than longitudinal strips of the second circumferential row of longitudinal strips (e.g., more of the side wall 146 remains between the longitudinal strips of the second circumferential row of longitudinal strips than between the longitudinal strips of the first circumferential row of longitudinal strips).

In some embodiments, each longitudinal strip of the second circumferential row of longitudinal strips may be axially aligned with one longitudinal strip of the first circumferential row of longitudinal strips. In some embodiments, each longitudinal strip of the second circumferential row of longitudinal strips may cooperate and/or combine with one longitudinal strip of the first circumferential row of longitudinal strips to form longer longitudinal strips interrupted by the second circumferential ring. In some embodiments, the longer longitudinal slots interrupted by the second circumferential ring may alternate around the circumference of the delivery sheath 140 with shorter longitudinal strips formed by remaining strips of the first circumferential row of longitudinal strips not included in or used to form the longer longitudinal strips interrupted by the second circumferential ring.

In some embodiments, at least one longitudinal strip of the plurality of longitudinal strips 160 may be wider at a distal end of the at least one longitudinal strip than at a proximal end of the at least one longitudinal strip. In some embodiments, the at least one longitudinal strip of the plurality of longitudinal strips 160 may be stepped from the proximal end toward and/or to the distal end. In some embodiments, the at least one longitudinal strip of the plurality of longitudinal strips 160 may be tapered from the proximal end toward and/or to the distal end. Other configurations are also contemplated.

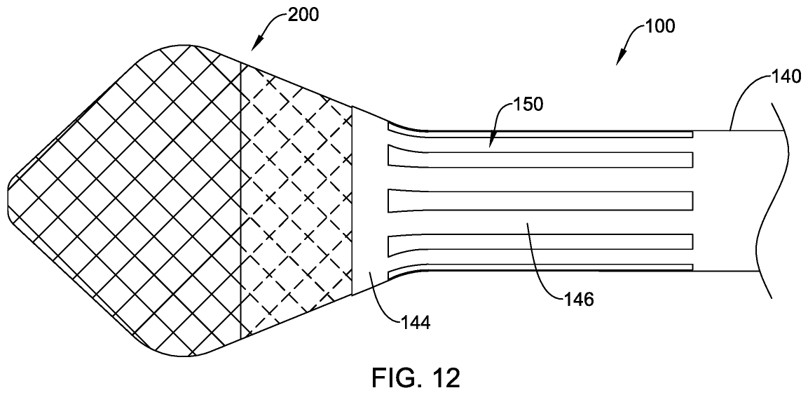

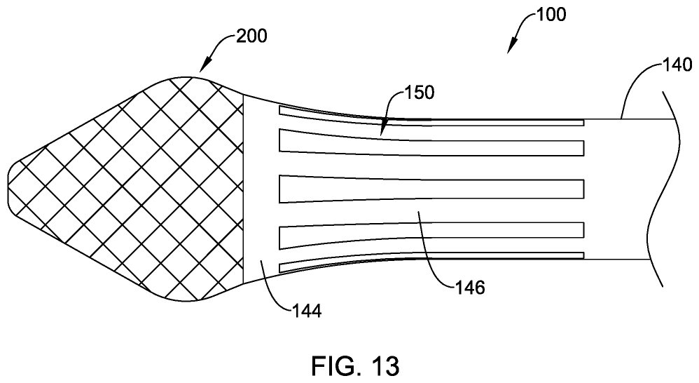

FIGS. 12-13 illustrates aspects of the delivery sheath 140 during recapture of the occlusive implant 200. As discussed herein, an access sheath may be used in conjunction with the occlusive implant system and/or the delivery system and thus may include some and/or all of the characteristics associated with the delivery sheath 140.

In use, after deploying the occlusive implant 200, imaging and/or other verification techniques may determine that the placement was inadequate or that the occlusive implant 200 needs to be repositioned and/or removed. In some embodiments, the delivery system may be used to recapture the occlusive implant 200 for removal and/or repositioning within the left atrial appendage. When pulling the occlusive implant 200 into the delivery lumen 142 of the delivery sheath 140 and shifting the expandable framework from the deployed configuration toward and/or to the delivery configuration (e.g., when collapsing the expandable framework), the distal end and/or the distal end region of the delivery sheath 140 may be configured to resiliently expand radially outward to prevent damage to the occlusive implant 200, the expandable framework 210, and/or the occlusive element 220, as well as the delivery sheath 140.

The plurality of longitudinal slots 150 may be configured to permit the distal end and/or the distal end region of the delivery sheath 140 to expand radially outward. By adding the plurality of longitudinal slots 150 to the delivery sheath 140, the delivery sheath 140 may be more flexible and/or may be permitted to elastically expand a greater amount and/or a greater magnitude than if the plurality of longitudinal slots 150 was not present. If the delivery sheath 140 is not permitted to expand radially outward a sufficient amount, the delivery sheath 140 may fail under the forces applied during recapture, such as by cracking, splitting, and/or splaying, thereby creating potentially hazardous features on the delivery sheath 140 within the patient's anatomy.

During recapture of the occlusive implant 200, as seen in FIGS. 12-13, the circumferential ring 144, the side wall 146, and/or the plurality of longitudinal slots 150 (which may include different configurations and/or arrangements as described herein) may stretch and/or expand radially outward and/or circumferentially. The distal end of the delivery sheath 140 and/or the circumferential ring 144 may be configured to expand a greater amount and/or a greater magnitude than a remainder of the delivery sheath 140. In some embodiments, the distal end and/or a distal portion of the plurality of longitudinal slots 150 may be configured to expand a greater amount and/or a greater magnitude than the proximal end and/or a proximal portion of the plurality of longitudinal slots 150. In some embodiments, the delivery sheath 140 may be stiffer, less clastic, and/or less flexible in a proximal direction. In some embodiments, the distal end of the delivery sheath 140 may be more elastic and/or more flexible and/or less stiff than a remainder of the delivery sheath 140.

The materials that can be used for the various components of the occlusive implant system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the delivery system, the delivery sheath, the occlusive implant, the core wire, the expandable framework, the occlusive clement, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 304 and/or 316 stainless steel and/or variations thereof; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

In at least some embodiments, portions or all of the system and/or other elements disclosed herein may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location and/or orientation of the system and/or other elements disclosed herein. Some examples of radiopaque materials can include, but are not limited to, gold, platinum, palladium, tantalum, tungsten alloy, polymer material loaded with a radiopaque filler, and the like.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A delivery system for an occlusive implant, comprising:
a delivery sheath having a delivery lumen extending proximally from a distal end of the delivery sheath, the delivery sheath being formed from a polymeric material; and
a core wire slidably disposed within the delivery lumen, the core wire being configured to releasably engage the occlusive implant;
wherein the delivery sheath includes a circumferential ring monolithically formed therewith at the distal end of the delivery sheath and a plurality of longitudinal slots disposed proximal of the circumferential ring.

2. The delivery system of claim 1, wherein the plurality of longitudinal slots is disposed adjacent the circumferential ring.

3. The delivery system of claim 1, wherein the plurality of longitudinal slots extends partially through a side wall of the delivery sheath.

4. The delivery system of claim 1, wherein the plurality of longitudinal slots extends completely through a side wall of the delivery sheath.

5. The delivery system of claim 4, wherein a second polymeric material different from the polymeric material is disposed within at least one longitudinal slot of the plurality of longitudinal slots.

6. The delivery system of claim 1, wherein the plurality of longitudinal slots comprises a first plurality of longitudinal slots having a first length and a second plurality of longitudinal slots having a second length different from the first length.

7. The delivery system of claim 6, wherein the first plurality of longitudinal slots alternates with the second plurality of longitudinal slots around a circumference of the delivery sheath.

8. The delivery system of claim 1, wherein the plurality of longitudinal slots comprises a first circumferential row of longitudinal slots and a second circumferential row of longitudinal slots disposed proximal of the first circumferential row of longitudinal slots.

9. The delivery system of claim 8, wherein adjacent longitudinal slots of the second circumferential row of longitudinal slots are spaced apart circumferentially a greater distance than adjacent longitudinal slots of the first circumferential row of longitudinal slots.

10. The delivery system of claim 8, wherein the first circumferential row of longitudinal slots comprises a first quantity of longitudinal slots and the second circumferential row of longitudinal slots comprises a second quantity of longitudinal slots less than the first quantity of longitudinal slots.

11. The delivery system of claim 8, wherein each longitudinal slot of the second circumferential row of longitudinal slots is axially aligned with one longitudinal slot of the first circumferential row of longitudinal slots.

12. The delivery system of claim 8, wherein the delivery sheath further comprises a second circumferential ring monolithically formed with the delivery sheath;
wherein the second circumferential ring is disposed axially between the first circumferential row of longitudinal slots and the second circumferential row of longitudinal slots.

13. The delivery system of claim 1, wherein at least one longitudinal slot of the plurality of longitudinal slots is wider at a distal end of the at least one longitudinal slot than at a proximal end of the at least one longitudinal slot.

14. The delivery system of claim 13, wherein the at least one longitudinal slot is tapered from the proximal end to the distal end.

15. An occlusive implant system, comprising:
an occlusive implant configured to shift between a delivery configuration and a deployed configuration; and
a delivery system for the occlusive implant, comprising:
a delivery sheath having a delivery lumen extending proximally from a distal end of the delivery sheath, the delivery sheath being formed from a polymeric material; and
a core wire slidably disposed within the delivery lumen, the core wire being releasably engaged with the occlusive implant at a distal end of the core wire;
wherein the delivery sheath includes a circumferential ring monolithically formed therewith at the distal end of the delivery sheath and a plurality of longitudinal slots disposed proximal of the circumferential ring;
wherein the occlusive implant is disposed within a distal portion of the delivery lumen in the delivery configuration.

16. The occlusive implant system of claim 15, wherein the occlusive implant comprises an expandable framework and an occlusive element secured to the expandable framework.

17. The occlusive implant system of claim 15, wherein the distal end of the delivery sheath is configured to expand radially outward during recapture of the occlusive implant.

18. The occlusive implant system of claim 15, wherein the circumferential ring extends uninterruptedly around the delivery lumen.

19. A delivery system for an occlusive implant, comprising:
a delivery sheath having a delivery lumen extending proximally from a distal end of the delivery sheath, the delivery sheath being formed from a polymeric material; and
a core wire slidably disposed within the delivery lumen, the core wire being configured to releasably engage the occlusive implant;
wherein the delivery sheath includes a circumferential ring monolithically formed therewith at the distal end of the delivery sheath, and a plurality of longitudinal strips formed from a second polymeric material different from the polymeric material;
wherein the plurality of longitudinal strips is embedded within a side wall of the delivery sheath proximal of the circumferential ring.

20. The delivery system of claim 19, wherein the second polymeric material has a lower durometer than the polymeric material.
